(19) United States
(12) Patent Application Publication
(10) Pub. No.: US 2006/0188866 A1
(43) Pub. Date: Aug. 24, 2006

(54) Diaminophenothiazine compositions and uses thereof

(75) Inventors: Hani Atamna, Albany, CA (US); Bruce N. Ames, Berkeley, CA (US)

Correspondence Address: RICHARD ARON OSMAN;SCIENCE AND TECHNOLOGY LAW GROUP, 242 AVE VISTA DEL OCEANO, SAN CLEMEMTE, CA, 92672, US

(73) Assignee: Children's Hospital Oakland Research Institute

(21) Appl. No.: 11/062,226

(22) Filed: Feb. 18, 2005

(51) Int. Cl. A01N 1/02; A61K 31/5415

(52) U.S. Cl. 435/2; 514/224.8

(57) ABSTRACT

Cell senescence is delayed by contacting a cell specifically determined to be in need of delayed cell senescence with an effective amount of a diaminophenothiazine.

### BACKGROUND OF THE INVENTION

Since it was first synthesized in 1876, methylene blue has been used for a variety of medicinal purposes. For example, it has been reported that bipolar manic-depressive patients treated with 300 mg/day methylene blue for one year were significantly less depressed than when treated with 15 mg/day placebo (Naylor, 1986). As another example, methylene blue at dosages of 65 mg taken three times a day has been reported to be useful in the management of chronic renal calculous disease (Smith, 1975). It has also been reported that methylene blue, injected at a dose of 1 mg/kg, improved brain oxidative metabolism and memory retention in rats (Callaway, 2004), and that senescence-enhanced oxidative stress is associated with deficiency of mitochondrial cytochrome c oxidase in vascular endothelial cells (Xin 2003).

We have found that methylene blue and related diaminophenothiazines, also sometimes called thiazins, can protect cells from oxidative stress and delay cell senescence at effective concentrations that are orders of magnitude lower than previously reported therapeutic doses of methylene blue.

### SUMMARY OF THE INVENTION

One aspect of the invention is a method for delaying cell senescence, the method comprising the step of: contacting a cell specifically determined to be in need of delayed cell senescence with an effective amount of a diaminophenothiazine; wherein the diaminophenothiazine has the structure: 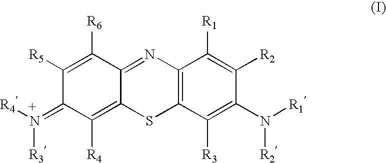
and tautomeric forms thereof, wherein R₁, R₂, R₃, R₄, R₅, R₆, R₁′, R₂′, R₃′, and R₄′ are independently hydrogen, methyl or ethyl; wherein the cell is a mitotically active cell in vitro in a culture medium comprising 1-100 nM diaminophenothiazine, and the contacting step comprises culturing the cell in the medium for at least one week.

In one embodiment, R₁, R₂, R₃, R₄, R₅, and R₆ are hydrogen, and R₂, R₅, R₁′, R₂′, R₃′, and R₄′ are independently hydrogen or methyl.

In another embodiment, the diaminophenothiazine is selected from the group consisting of azure A, azure B, azure C, thionine, toluidine blue, methylene blue, new methylene blue, and 1-9-dimethyl methylene blue.

In one embodiment, the culture medium comprises 10-100 nM methylene blue.

In one embodiment the contacting step comprises culturing the cell in the medium for at least 4 weeks.

In another embodiment, the cell is additionally contacted with one or more mitochondrial protective agent selected from the group consisting of an N-hydroxylamine, acetyl carnitine, and lipoic acid.

Another aspect of the invention is a method for delaying cell senescence, the method comprising the step of: contacting a cell specifically determined to be in need of delayed cell senescence with an effective amount of a diaminophenothiazine; wherein the diaminophenothiazine has the structure of Formula I and tautomeric forms thereof, wherein R₁, R₂, R₃, R₄, R₅, R₆, R₁′, R₂′, R₃′, and R₄′ are independently hydrogen, methyl or ethyl; wherein the cell is in situ in an individual, and the contacting step comprises chronically orally administering to the individual a dosage of 5-500 μg per day of the diaminophenothiazine to provide said effective amount.

In one embodiment R₁, R₂, R₃, R₄, R₅, and R₆ are hydrogen, and R₂, R₅, R₁′, R₂′, R₃′, and R₄′ are independently hydrogen or methyl.

In another embodiment the diaminophenothiazine is selected from the group consisting of azure A, azure B, azure C, thionine, toluidine blue, methylene blue, new methylene blue, and 1-9-dimethyl methylene blue.

In one embodiment the dosage is administered daily for at least 30 days.

In one embodiment the individual is over 40 years old.

In another embodiment the individual is free of diagnosed acute disease or pathology.

In one embodiment, the contacting step additionally comprises chronically orally administering to the individual one or more mitochondrial protective agent selected from the group consisting of an N-hydroxylamine, acetyl carnitine, and lipoic acid.

Another aspect of the invention is a pharmaceutical composition comprising a unit dosage for oral administration of 5 to 500 μg of a diaminophenothiazine and a pharmaceutically acceptable excipient, wherein the diaminophenothiazine has the structure of Formula I or a tautomeric form thereof, wherein R₁, R₂, R₃, R₄, R₅, R₆, R₁′, R₂′, R₃′, and R₄′ are independently hydrogen, methyl or ethyl.

In one embodiment R₁, R₂, R₃, R₄, R₅, and R₆ are hydrogen, and R₂, R₅, R₁′, R₂′, R₃′, and R₄′ are independently hydrogen or methyl.

In another embodiment the diaminophenothiazine is selected from the group consisting of azure A, azure B, azure C, thionine, toluidine blue, methylene blue, new methylene blue, and 1-9-dimethyl methylene blue.

In another embodiment, the composition is packaged with a label identifying the diaminophenothiazine and prescribing a pharmaceutical use thereof and the use comprises delaying cell senescence.

In another embodiment the composition additionally comprises one or more mitochondrial protective agent selected from the group consisting of an N-hydroxylamine, acetyl carnitine, and lipoic acid.

Another aspect of the invention is a method for marketing the pharmaceutical composition comprising promoting use of an effect amount of a diaminophenothiazine to delay cell senescence in an individual determined to have cells in need of delayed cell senescence.

Another aspect of the invention is a cell culture medium comprising 1-100 nM of a diaminophenothiazine, wherein the diaminophenothiazine has the structure of formula I or a tautomeric form thereof, wherein R₁, R₂, R₃, R₄, R₅, R₆, R₁′, R₂′, R₃′, and R₄′ are independently hydrogen, methyl or ethyl.

In one embodiment R₁, R₂, R₃, R₄, R₅, and R₆ are hydrogen, and R₂, R₅, R₁′, R₂′, R₃′, and R₄′ are independently hydrogen or methyl.

In another embodiment the diaminophenothiazine is selected from the group consisting of azure A, azure B, azure C, thionine, toluidine blue, methylene blue, new methylene blue, and 1-9-dimethyl methylene blue.

In another embodiment, the cell culture medium additionally comprises one or two or three mitochondrial protective agent selected from the group consisting of an N-hydroxylamine, acetyl carnitine, and lipoic acid.

### DETAILED DESCRIPTION OF SPECIFIC EMBODIMENTS OF THE INVENTION

The invention provides a method for delaying cell senescence comprising the step of: contacting a cell specifically determined to be in need of delayed cell senescence with an effective amount of a diaminophenothiazine.

Diaminophenothiazines of the invention have the structure 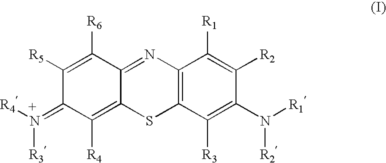
and tautomeric wherein R₁, R₂, R₃, R₄, R₅, R₆, R₁′, R₂′, R₃′, and R₄′ are independently hydrogen, methyl or ethyl.

Diaminophenothiazines are positively charged due to electron delocalization resulting in partial positive charges located on both nitrogen and sulfur atoms. Scheme A shows the chemical structure of thionine, which is the simplest diaminophenothiazine, and the three tautomeric forms of thionine created by electron delocalization:

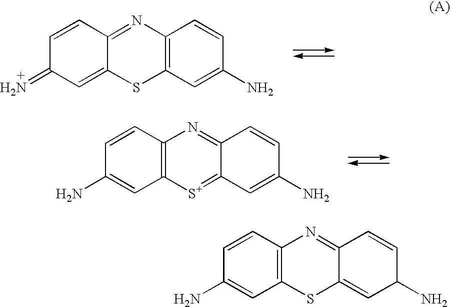

In one embodiment of the invention, the diaminophenothiazine has the structure of formula I, wherein R₁, R₂, R₃, R₄, R₅, and R₆ are hydrogen, and R₂, R₅, R₁′, R₂′, R₃′, and R₄′ are independently hydrogen or methyl. In another embodiment of the invention, the diaminophenothiazine is selected from the group consisting of azure A, azure B, azure C, thionine, toluidine blue, methylene blue, new methylene blue, and 1-9-dimethyl methylene blue.

The diaminophenothiazine is administered to a cell in an amount sufficient to delay cell senescence, preferably a dose-minimized effective amount. In the case of a mitotic cell, delaying cell senescence prolongs the duration in which the cell remains mitotically active, resulting in an increased number of population doublings relative to age-matched control cells not treated with the diaminophenothiazine. In the case of quiescent cells, delayed cell senescence results in reduced oxidative stress or increased cell vitality or viability, relative to age-matched control cells not treated with the diaminophenothiazine. Dose-minimized amounts of diaminophenothiazine effective in delaying cell senescence is typically in the range of about 1 to 100 nM, depending on the cell type and status, culture conditions, and selected diaminophenothiazine. Optimal amounts of a diaminophenothiazine for a particular cell, tissue, or organ can be empirically determined using routine experimentation, such as described in Example 1.

A cell specifically determined to be in need of delayed cell senescence can be any cell for which there is an interest in delaying senescence. As used herein, the singular forms “a,” “an,” and “the,” refer to both the singular as well as plural, unless the context clearly indicates otherwise. For example, the term “a cell” includes single or plural cells and can be considered equivalent to the phrase “at least one cell.” In preferred embodiments, the cell is an animal cell, preferably a mammalian cell, more preferably a human cell.

In one embodiment of the invention, the cell is a mitotically active cell in vitro in a culture medium. Delaying senescence of cultured cells may be needed to increase the productivity of culture methods, resulting in a greater number of cells for a particular use and/or to prolong the duration in which a cell has adequate health and vitality for its intended use. The cells may be undifferentiated, differentiating, and/or differentiated. In one embodiment, the cells are used for heterologous or autologous cell transplantation therapy, such as fibroblasts and/or keratinocytes used for artificial skin, embryonic or adult progenitor or stem cells, neural cells, hematopoietic cells, bone marrow cells, myocytes, etc. In particular embodiments, particular for stem cells, culture conditions comprise 0.1-20%, preferably 1-20%, more preferably 5-20% oxygen; and/or 1-20 mM, preferably 5-20 mM glucose.

The cells may be cultured in a diaminophenothiazine-supplemented medium for as long as necessary to achieve the desired degree of delayed cell senescence. In one embodiment, the cells are continuously cultured in the diaminophenothiazine-supplemented medium, and passaged as needed, for at least 1 week. In further embodiments, the cells are cultured in a diaminophenothiazine-supplemented medium for at least 2, 4, or 8 weeks. In one embodiment, the cells are continuously cultured in a diaminophenothiazine-supplemented medium for at least 12 weeks. In another embodiment, the culture medium comprises 1-100 nM diaminophenothiazine, and the contacting step comprises culturing the cell in the medium for at least one week.

The method of the invention may further comprise the step of detecting a resultant delay in cell senescence. For cells in vitro, a delay in cell senescence can be directly detected by measuring an increase in duration of mitotic activity and/or life-span of diaminophenothiazine-treated cells compared to age-matched controlled cells not treated with the diaminophenothiazine, such as described in Example 1. A resultant delay in cell senescence can also be inferentially detected, for example by measuring, relative to aged-matched control cells, increases in cell vitality, for example by using a commercially available cell vitality assay kit (e.g. Invitrogen, Carlsbad Calif.); markers of oxidative stress such as 8-hydroxy-2′-deoxyguanosine, glutathione (see Atamna, 2001), or nitrotyrosine (e.g. OxisResearch, Portland, Oreg.); or detection of a delay in age-dependent changes in mitochondria such as accumulation of rhodamine-123, reduced cytochrome c oxidase activity, and decay of mitochondrial aconitase (see Atamna, 2000).

In one embodiment of the invention, the cell is additionally contacted with one or more mitochondrial protective agent in a mitochondrial protective, preferably dose-minimized amount. Such agents include N-hydroxylamines (e.g., Atamna, 2000; Ames, 2002; U.S. Pat. No. 6,455,589), acetyl carnitine, and lipoic acid (e.g. Hagen, 2000); effective, dose-minimized amounts are readily determined from art-recognized literature, such as the publications cited herein, and/or empirically, using assays disclosed therein. By way of example, mitochondrial protective dosages of N-hydroxylamines in humans will typically be from about 100 ug to 1 g, preferably from about 10 ug to 1 g, more preferably at least 100 ug, more preferably at least 1 mg, more preferably at least 10 mg, most preferably at least 100 mg. In one embodiment, the cell is additionally contacted with acetyl carnitine and lipoic acid (e.g. U.S. Pat. No. 5,916,912). By way of example, mitochondrial protective dosages of this combination will typically be from about 1-50 mg/kg host/day carnitine together with about 1-50 mg/kg host/day of lipoic acid, preferably about 10 mg/kg host/day carnitine together with about 10 mg/kg host/day lipoic acid.

Another aspect of the invention is a cell culture medium comprising about 1-100 nM of a diaminophenothiazine, wherein the diaminophenothiazine has the structure of formula I or a tautomeric form thereof, wherein R₁, R₂, R₃,R₄, R₅, R₆, R₁′, R₂′, R₃′, and R₄′ are independently hydrogen, methyl or ethyl. In one preferred embodiment, R₁, R₂, R₃, R₄, R₅, and R₆ are hydrogen, and R₂, R₅, R₁′, R₂′, R₃′, and R₄′ are independently hydrogen or methyl. In another embodiment, the diaminophenothiazine is selected from the group consisting of azure A, azure B, azure C, thionine, toluidine blue, methylene blue, new methylene blue, and 1-9-dimethyl methylene blue. Any cell culture medium can be supplemented with the diaminophenothiazine, e.g. MEM, DMEM, RPMI-1640, Ham's F-10 and F-12 media, etc. The cell culture medium may additionally comprise one or more mitochondrial protective agent selected from the group consisting of an N-hydroxylamine, acetyl carnitine, and lipoic acid. In one embodiment the cell culture medium additionally comprises acetyl carnitine and lipoic acid.

In another embodiment of the invention, the cell is in situ in an individual and the contacting step comprises chronically orally administering to the individual a dosage of 5-500 μg per day of the diaminophenothiazine to provide the effective amount. Delaying senescence of cells in situ may be desired for delaying the effects associated with normal or premature aging. For example, fibroblast senescence in human skin is associated with signs of aging such as wrinkling and loss of elasticity caused by reduced collagen production. Further, age-related changes in mitochondria are associated with reduced amubulatory acitivity (e.g. Atamna, 2001). In certain embodiments, the individual is over 40 years old. In further embodiments the individual is over 50, 65, or 70 years old. In another embodiment, the individual is diagnosed to have a mitochondrial-associated disease, particularly a mitochondrial-associated disease neuropathy, cardiomyopathy, or encephalopathy, such as MELAS, MERRF, NARP, Myoneurogastrointestinal disorder and encephalopathy (MNGIE), Pearson Marrow syndrome, Kearns-Sayre-CPEO, Leber hereditary optic neuropathy (LHON), Aminoglycoside-associated deafness, Diabetes with deafness, etc. In another embodiment, the individual is diagnosed to have a chronic neurodegenerative disease, such as Alzheimer's Disease, Huntington's Disease, Parkinson's Disease, etc., or an acute neurodegenerative condition such as stroke. In another embodiment, the individual has or is diagnosed to have insulin resistance, or diabetes, particularly type II diabetes. In another preferred embodiment, the individual is free of diagnosed acute disease or pathology, particularly diseases and pathologies in which diaminophenothiazine therapy is specifically indicated or contraindicated. Examples of such diseases or pathologies include cancers being treated with ifosfamide and methylene blue combination therapy (Aeschlimann, 1998), chronic renal calculous disease (Smith, 1975), manic-depressive psychosis (Naylor, 1986), and malaria (Vennerstrom, 1995).

In one embodiment, the dosage is between 0.1-10.0 μg/kg body weight/day. In a preferred embodiment, the dosage achieves a blood concentration of about 1.0 to 100 nM of the diaminophenothiazine. In another embodiment, the dosage achieves a brain concentration of about 1.0 to 100 nM of the diaminophenothiazine. For a human weighing about 160 lbs and having about 5 liters of blood, the daily dose of methylene blue (mol. wt.=319) needed to achieve this concentration is about 1.6 to 160 μg. The oral dosage is typically given one to three times daily for prolonged periods, usually for at least 30 days. In some cases, the oral dosage is administered chronically, i.e. regularly over a period of at least 3, preferably at least 6, more preferably at least 12 months, wherein the regularity is at least once, preferably at least 2-3 times, more preferably at least 7 times (daily) per week. In particular embodiments, the compositions are administered once, twice, thrice or four times per day. In further embodiments, the individual is additionally chronically orally administered a mitochondrial protective agent such as an N-hydroxylamine, acetyl carnitine, or lipoic acid. In another embodiment, the individual is additionally chronically orally administered acetyl carnitine and lipoic acid.

In certain embodiments, the method further comprises the step of detecting a resultant delay in cell senescence in the individual. For cells in situ, delayed cell senescence is typically detected inferentially, for example by detecting increases in cell vitality, such as improved elasticity or increased skin thickness relative to pretreatment; a reduction in the levels of markers of oxidative stress; delay in age-dependent changes in mitochondria such as increased cytochrome c oxidase activity or levels; and/or increased ambulatory activity in the individual.

Another aspect of the invention is a pharmaceutical composition comprising a unit dosage for oral administration of 5 to 500 μg of a diaminophenothiazine and a pharmaceutically acceptable excipient, wherein the diaminophenothiazine has the structure of Formula I, or a tautomeric form thereof, wherein R₁, R₂, R₃, R₄, R₅, R₆, R₁′, R₂′, R₃′, and R₄′ are independently hydrogen, methyl or ethyl. In one embodiment, R₁, R₂, R₃, R₄, R₅, and R₆ are hydrogen, and R₂, R₅, R₁′, R₂′, R₃′, and R₄′ are independently hydrogen or methyl. In another embodiment, the diaminophenothiazine is selected from the group consisting of azure A, azure B, azure C, thionine, toluidine blue, methylene blue, new methylene blue, and 1-9-dimethyl methylene blue. In a preferred embodiment, the diaminophenothiazine is methylene blue. In a further embodiment, the diaminophenothiazine is methylene blue and the unit dosage is selected from 200 μg, 150 μg, 100 μg, 50 μg, 40 μg, 25 μg, and 10 μg. The oral dosages can be prepared in any form suitable for oral administration, including tablets, capsules, lozenges, troches, hard candies, powders, metered sprays, creams, suppositories, etc. The composition is combined with a pharmaceutically acceptable excipient such as gelatin, an oil, etc. and may include additional active agents. In one embodiment, the pharmaceutical composition additionally comprises a mitochondrial protective agent selected from the group consisting of an N-hydroxylamine, acetyl carnitine, and lipoic acid. In another embodiment, the composition additionally comprises acetyl carnitine and lipoic acid.

In one embodiment, the pharmaceutical composition of the invention is packaged with a label identifying the diaminophenothiazine and prescribing its use for reducing oxidative damage and/or delaying cell senescence.

Another aspect of the invention is a method for marketing an above-described diaminophenothiazine, or any of the above-described pharmaceutical compositions, wherein the method comprises promoting use of an effective amount of a diaminophenothiazine to delay cell senescence in an individual determined to have cells in need of delayed cell senescence. In one embodiment, the individual is over 40 years old. In a further embodiment, the individual is desirous of taking a supplement to reduce the signs of aging skin by improving skin elasticity and/or reducing wrinkles, and/or increasing energy levels, and the diaminophenothiazine is promoted for such use. Any conventional media may be used for the marketing such as product labels, package inserts, internet marketing, television commercials, newspaper and magazine articles, etc. The marketing will typically include a description of the use of the diaminophenothiazine to delay cell senescence and potential health benefits of such use, such as improved skin elasticity, wrinkle reduction, and/or increased energy levels.

### EXAMPLE 1

### Diaminophenothiazines Delay Senescence of Human Lung Fibroblast Cells (IMR₉₀) in Tissue Culture

Normal human epithelial fibroblast (IMR₉₀) cells were obtained from the Coriell Institute for Medical Research at a population doubling level (PDL) of 10.85. The PDLs were calculated as log₂(D/Dₒ), where D and Dₒ are defined as the density of cells at the time of harvesting and seeding, respectively. Stock cultures were grown in 100-mm Corning tissue culture dishes containing 10 ml of Dulbecco's modified Eagle's medium supplemented with 10% (V/V) fetal bovine serum (Hyclone). Stock cultures were split once a week when near confluence. Cells were harvested by trypsinization for 5 min at 37° C., immediately collected in 5 ml of complete Dulbecco's modified Eagle's medium, washed once with 5 ml of complete Dulbecco's modified Eagle's medium, and incubated for 10-15 min at 37° C. to allow the cells to recover.

To test the effect of diaminophenothiazines on replicative life span, IMR₉₀ cells were seeded in air at 0.5×10⁶/100-mm dish. Diaminophenothiazines (methylene blue or thionine) were added to the culture medium at a concentration of 10 nM, 100 nM, or 100 nM. The cultures were harvested every 7 days, counted, and PDL were calculated. A portion of the cells were used to seed new dishes in fresh medium (control) or fresh medium supplement with the diaminophenothiazines described above. The culture conditions and methods were repeated using the same concentrations of methylene blue (and control conditions), except that the cells were cultured at 5% oxygen.

Methylene blue delayed the senescence of the IMR₉₀ cells seeded in air by at least 25-30 population doublings. The optimal concentration is between 10-100 nM. Thionine is also effective at 100 nM. Methylene blue also delayed senescence of the IMR₉₀ cells seeded in 5% oxygen. The increase in PDL in 5% oxygen ranges between 10-15. The oxygen concentration in air is about 20%, while 5% oxygen is about the physiologic concentration, and normally results in considerably more PDLs for human cells in culture.

The level of mitochondrial complex IV in the IMR₉₀ cells was determined using specific antibody for subunit II (a core subunit essential for the assembly of the entire complex), western blotting, and protein density measurements using NIH Image. Methylene blue increased the level of complex IV by approximately 30% above age-matched controls. The effect of methylene blue was bell-shaped and at 1000 nM, methylene blue caused removal of complex IV from the mitochondria. An increase in complex IV was also seen after treatment with thionine.

In another experiment, the effect of hydrogen peroxide (H₂O₂) on senescence of methylene blue-treated cells was tested. IMR₉₀ cells were seeded at an initial density of 0.5×10⁶/dish and grown for two weeks in the presence of methylene blue prior to H₂O₂ treatment. Thereafter, cells were harvested weekly, counted, and seeded with or without methylene blue and/or 10 μM H₂O₂. Cells that were treated with hydrogen peroxide without methylene blue treatment had accelerated senescence. The H₂O₂-induced senescence was prevented when these cells were continuously treated with 100 nM methylene blue.

### EXAMPLE 2

### Diaminophenothiazines Delay Senescence of Expanded Umbilical-Cord Blood Hematopoietic Stem Cells

Human umbilical cord blood (UCB) cells have become an attractive source of hematopoietic precursors for allogeneic stem cell transplantation in children with inborn errors or malignant diseases. One major advantage of UCB cells in comparison with peripheral blood stem cells or bone marrow (BM) is the reduced incidence of acute graft-versushost disease caused by cord blood grafts. Although UCB cells are easy to collect and store, the usage into transplantation protocols for adults has been restricted by the limited number of progenitors contained in one cord blood harvest. The prolonged time to engraftment as well as the existence of very early hematopoietic progenitors have led hematologists to develop ex vivo expansion protocols for UCB stem cells for clinical use.

One of the major challenges in stem cell research is to increase transplantable HSC number by stimulating self-renewal divisions of hematopoietic stem cells ex vivo in order to expand the primitive compartment and precursor cells. UCB cells exhibit high proliferative capacities leading within few weeks to a large expansion of cells in response to various combinations of hematopoietic growth factors. Optimal combination of different media as well as different growth factor cocktails have resulted in efficient expansion of progenitors, but amplification of long-term culture initiating cells has been more challenging.

In a series of experiments adapted from Chivu et al. (J Cell Mol Med. 2004 April-June 8(2):223-3 1), we demonstrate beneficial use of diaminophenothiazines in several culture protocols for in vitro maintenance of umbilical cord blood stem cells. Our results indicate that PCM and primary BM cultures supplemented with diaminophenothiazines provide improved support for growth of hematopoietic cells in vitro and maintaining long-term culture initiating cells being inoculated as mononuclear cell (MNC) fractions.

Cells. Blood is collected according to institutional guidelines during normal and routine full-term deliveries, using citrate phosphate dextrose adenine as anticoagulant. Mononuclear cells (MNC) are separated on Histopaque (Sigma); density gradient=1.007 g/ml), resuspended in Iscove's modified Dulbecco's medium (IMDM) and counted.

Expansion cultures. Low-density MNC are resuspended in serum-free culture medium at a final concentration of 2×106 cells per ml, and with medium composition as follows: Dulbecco medium supplemented with 1% bovine serum albumin (Merck), 5×10−4 M 2-mercaptoethanol, 1% non-essential amino acids (Gibco Life-Sciences), 2 mM L-glutamine and freshly dissolved 10⁻⁶ M hydrocortisone. Cell suspensions are loaded into 24-well plates (1 ml/well) and incubated at 37° C. in an atmosphere of 5% CO2. Human growth factors are used at concentrations providing highest cell proliferation in titration experiments: 10 ng/ml interleukin 6 (Chemicon), 20 ng/ml G-CSF (Hoffmann-La Roche), and 2 U/ml erythropoietin (Hoffmann-La Roche) are used. A second growth cocktail is based on 20% placental conditioned medium (PCM) (obtained as described before—Chivu et al. , J. Cell. Mol. Med., 6: 609-620, 2002) and 2% autologous plasma. Each of eight diaminophenothiazines (azure A, azure B, azure C, thionine, toluidine blue, methylene blue, new methylene blue, and 1-9-dimethyl methylene blue) are separately tested in each of three serial concentrations: 1 nM, 10 nM, and 100 nM. After three days, total numbers of nucleated and viable cells are determined using trypan blue exclusion test.

Establishment of feeder layer. For generation of preformed stroma, long-term bone marrow culture is established using mouse bone marrow cells. The marrow is mixed with the same volume of PBS, and mononuclear cells separated by density gradient centrifugation. Washed cells are resuspended in stromal medium: F12 medium (Sigma) with 10% fetal calf serum, 400 UI/ml penicillin, 200 mg/ml streptomycin and 1.0 μmol/l hydrocortisone. Cells are maintained at 37° C. and 5% CO2, with half the medium exchanged twice a week. After two weeks, cells from confluent adherent layers are recovered by digestion with trypsin-EDTA solution (Sigma-Aldrich) and transferred to six-well plates for further experiments.

Cultures are incubated at 37° C. in an atmosphere of 5% CO2. When a confluent stroma layer is formed, cultures are treated with 20 μg/ml mitomycin C (Sigma) for 4 h, then washed extensively to remove the drug. At the end cells are viable and metabolically active, but blocked in cell cycle. Additional cultures supplemented with growth factors and PCM are similarly established.

Flow cytometric analysis. Acquisition of labelled cells is performed using a FACS can flow cytometer (Becton Dickinson) Control and induced cells are collected, washed twice in PBS, 0.1% BSA, 0.1% sodium azide at 4° C. and finally fixed in 1% paraformaldehyde. Analysis of all samples before and after expansion is performed using monoclonal antibodies against CD34, CD 14, CD45, CD71, HLA-DR (Becton Dickinson) to categorize progenitor subsets. Ten thousand events are acquired and data analyzed with WinMDI. CD34+ cells are determined as percentages of lymphocyte gate (set on CD45+ low side scatter cells and maintained throughout analysis, excluding CD14+ cells).

Semisolid clonal assays. 1×105 UCB cells are plated in a 0.3% agar medium containing IMDM with 30% fetal bovine serum, 10 ng/ml IL-6, 20 ng/ml G-CSF, 2 U/ml erythropoietin, 10-4 M/L 2- mercaptoethanol and 1% bovine serum albumin. Dishes are incubated at 37° C. and 5% CO2 in a humidified atmosphere. Cultures are assessed after 14 days for the presence of burst-forming unit-erythroid (BFU-E), colony forming-unit granulocyte/macrophage (CFU-GM), and mixed colony-forming unit (CFU-GEMM).

Statistical analysis. For most parameters, median and range are provided. Experiments evaluating different conditions are compared using the t-test for paired samples. Statistical significance is assumed when the two-tailed p value was below 0.05.

Expansion efficiency of CD34+ cells. Cultures of MNC from umbilical cord blood are initiated in the presence of various growth conditions such as feeder cell layer, supplement of exogenous cytokines cocktail (IL-6, G-CSF, erythropoietin) or PCM. The expansion efficiency is expressed as the fold expansion of various cell subsets, where the fold expansion is the increase of the cells relative to the control untreated cells.

Flow cytometric analysis of CD34+ populations demonstrate a clear benefit of using diaminophenothiazines at each of the assayed nanomolar concentrations in PCM in combination with feeder layer during standard cytokine-based in vitro UCB expansion conditions. Results showed an increase of CD34+ cell population, and long-term culture initiating cells number, above control cells. Diaminophenothiazine-treated cultures consistently demonstrate an increased number of population doublings relative to age-matched control cells not treated with the diaminophenothiazine.

### EXAMPLE 3

### Diaminophenothiazines Delay Senescence of Expanded Human Embryonic Stem Cells

In a parallel series of experiments, we demonstrate beneficial use of the same diaminophenothiazines in culture protocols for in vitro maintenance of human embryonic stem cells. In a protocol adapted from Schuldiner, et al. (2000, Proc. Natl. Acad. Sci. USA 97, 11307-11312), human ES cells (H9 clone (Thomson, et al.,1998, Science 282, 1145-1147)) are grown on mouse embryo fibroblasts in 80% KnockOut DMEM, an optimized Dulbecco's modified Eagle's medium for ES cells (GIBCO/BRL), 20% KnockOut SR, a serum-free formulation (GIBCO/BRL), 1 mM glutamine (GIBCO/BRL), 0.1 mM beta-mercaptoethanol (Sigma), 1% nonessential amino acids stock (GIBCO/BRL), 4 ng/ml basic fibroblast growth factor (bFGF) (GIBCO/BRL), and 10³ units/ml LIF (GIBCO/BRL).

As above, each of eight diaminophenothiazines (azure A, azure B, azure C, thionine, toluidine blue, methylene blue, new methylene blue, and 1-9-dimethyl methylene blue) are separately tested in each of four serial concentrations: 1 nM, 10 nM, and 100 nM. Diaminophenothiazine-treated cultures consistently demonstrate an increased number of population doublings relative to age-matched control cells not treated with the diaminophenothiazine.

### EXAMPLE 4

### Effect of diaminophenothiazines on Food Consumption and Ambulatory Activity in Old Rats, and Age-Related Oxidative Changes in their Livers

Methodology is adapted from Atamna et al. (2001). Young (age 3 months; Simonsen, Gilroy, Calif.) and old (24 months, National Institute of Aging animals colonies) male Fisher 344 rats are divided equally into control and diaminophenothiazine treatment groups. At commencement of the study, each treatment group consists of four or five rats housed together in large cages in order to minimize stress, in conditions of controlled temperature (25° C.) and a 12 h light/dark cycle (6:00 h to 18:00 h). The rats are allowed ad libitum access to standard Purina rodent chow. Methylene blue is administered to the rats in double distilled water at a final concentration of 100 nM for a period of 25 days, which, based on typical water consumption by rats, should provide approximately 1-2 μg/kg/d methylene blue. The salinity of the drinking water is adjusted to 1 μmol NaCl/ml and sodium hydroxide is used to adjust the water to pH 6 for all groups. Fresh water with or without methylene blue is supplied daily. Body weight is measured weekly and food and water intake is measured daily. Chow or water intake is measured at the beginning and end of every 24 h period and the difference is divided by the number of the animals in the cage. At the end of the experiment, the rats are anesthetized with ether and killed by cardiac puncture. The liver is resected and placed in ice-cold mitochondrial isolation buffer containing 210 mM mannitol, 70 mM sucrose, 5 mM HEPES, and 1 mM EDTA, pH 7 (MSH/EDTA). The liver is homogenized immediately and the mitochondrial fraction is isolated by differential centrifugation. Mitochondrial respiration supported by succinate 5 mM, phosphate (4 mM), and ADP (0.15 mM) is measured in 125 mM KCl and 5 mM Tris, pH 7.4 by a Clark Oxygen electrode (Yellow Springs Instruments, Yellow Springs, Ohio) in the presence of 4 μM rotenone.

This experimental protocol is repeated in a group of old rats (n=10) that are either untreated or administered methylene blue for 25 days. The results of the two experiments are pooled for data analysis.

To measure ambulatory activity, on day 21 of the study, rats are transferred to individual cages (48 cm long×25 cm wide×20 cm high) for measurements of ambulatory activity. Rats are acclimatized to their new surroundings for at least 4 h before monitoring. Rats have ad libitum access to food and water. The room is on a 12 h light/dark cycle (lights on 6:00 to 18:00). At 20:00 h, a very low intensity light illuminates the rats for video tracking. Monitoring of ambulatory activity begins at 21:00 h and continues for 4 h. One hour later, the low light is turned off and the standard light cycle is continued. The ambulatory activity of each rat is recorded for four consecutive nights. A video signal from a camera suspended directly above the individual cages is connected to a Videomex-V (Columbus Instruments, Columbus, Ohio) computer system running the Multiple Objects Multiple Zones software. The system quantifies ambulatory activity parameters and is calibrated to report distance traveled in centimeters.

To determine levels of free GSH and protein-mixed disulfides in the rat livers, a 200 μl aliquot of liver homogenate is immediately transferred into 50 μl of 1 M methane sulfonic acid (MSA) and 2.5 mM DTPA and stored at −80° C. until analysis. The proteins from the MSA homogenate are precipitated by centrifugation at high speed. The supernatant is used for quantification of free GSH. The pellet is washed three times by resuspending in ice-cold PBS. The final pellet is resuspended in 100 μl of ice-cold 0.1 M Tris and 50 mM DTT (pH 8.3) and incubated on ice. After 1 h incubation, 20 μl of 1 M MSA and 2.5 mM DTPA are added to precipitate the proteins and stabilize GSH. The pellet is used for protein quantification (Bio-Rad protein assay, Bio-Rad); the supernatant is filtered and used for quantification of the GSH that is liberated from the mixed disulfides in the proteins. Both supernatant are filtered through 30,000 cutoff filters before injection into an HPLC column. The amount of protein injected is 5-10 μg or 1-3 μg for GS-SR and free GSH, respectively. Free GSH and GSH liberated from protein-mixed disulfides after reduction by DTT is determined by HPLC-EC detection. The activities of glutamate dehydrogenase and glucose-6-phosphate dehydrogenase are assayed.

The above described experiments are repeated with the following additional diaminophenothiazines at varying concentrations to achieve blood concentrations of 0.1 nM to 100 nM: new methylene blue, 1-9-dimethyl methylene blue, and azure B. Diaminophenothiazine treatments that result in increased food consumption and ambulatory activity in old rats and/or a reduction in oxidative stress, as indicated by reduced levels of GSH in the liver, provide suitable reagents for human clinical trials.

### EXAMPLE 5

### Long-Term Administration of diaminophenothiazines Improve Skin Elasticity and Activity Levels in Patients over 50.

A double blind, randomized, vehicle controlled study is conducted in 160 subjects ranging in ages from 50 to 65. Treatment groups are prescribed 25 μg diaminophenothiazine (azure A, azure B, azure C, thionine, toluidine blue, methylene blue, new methylene blue, or 1-9-dimethyl methylene blue) tablets taken orally with meals 3 times daily. Clinical monitoring, subjective self assessment, objective measurement methods of skin elasticity, epidermal hydration and skin surface lipids are used to determine effects of each treatment at four visits during 24 weeks. Clinical monitoring includes wrinkle counts, measurement of wrinkle depth around the right eye, and nasolabial fold depth. Results demonstrate consistent efficacy of diaminophenothiazine treatment over placebo in counteracting different signs of aging in the skin and improving overall energy levels.

The foregoing examples and detailed description are offered by way of illustration and not by way of limitation. All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

### REFERENCES

Aeschlimann et al, “Comparative pharmacokinetics of oral and intravenous ifosfamide/mesna/methylene blue therapy.” Drug Metab Dispos. (1998) 26:883-90.
Ames and Atamna, (2002) “Primary N-Hydroxylamines” U.S. Pat. No. 6,455,589.
Ames and Hagen, (1999) “Dietary composition for enhancing metabolism and alleviating oxidative stress” U.S. Pat. No. 5,916,912.
Atamna et al, “A method for detecting abasic sites in living cells: age-dependent changes in base excision repair” Proc Natl Acad Sci U S A. (2000) 97:686-91.
Atamna et al, “N-t-Butyl hydroxylamine is an antioxidant that reverses age-related changes in mitochondria in vivo and in vitro.” FASEB J. (2001) October 15(12):2196-204.
Callaway et al, “Methylene blue improves brain oxidative metabolism and memory retention in rats.” Pharmacol Biochem Behav. (2004) 77:175-81.
Hagen et al, “Feeding acetyl-L-carnitine and lipoic acid to old rats significantly improves metabolic function while decreasing oxidative stress.” Proc Natl Acad Sci U S A. (2002) 99:1870-5.
Naylor et al., “A two-year double-blind crossover trial of the prophylactic effect of methylene blue in manic-depressive psychosis.” Biol Psychiatry 1986 August 21(10):915-20.
Smith M J, “Methylene blue in renal calculi. Results of five-year study” Urology (1975) 6:676-9.
Vennerstrom et al, “Antimalarial dyes revisited: xanthenes, azines, oxazines, and thiazines” Antimicrob Agents Chemother. (1995) 39:2671-7.
Xin et al., Sensecence-enhanced oxidative stress is associated with deficiency of mitochondrial cytochrome c oxidase in vascular endothelial cells., Mech Ageing Dev. 2003 August-September; 124(8-9):911-9.

1. A method for delaying cell senescence, the method comprising the step of: contacting a cell specifically determined to be in need of delayed cell senescence with an effective amount of a diaminophenothiazine; wherein the diaminophenothiazine has the structure: 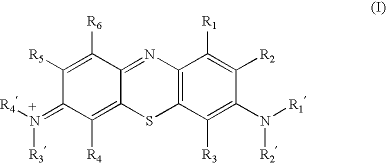
and tautomeric forms thereof, wherein R₁, R₂, R₃, R₄, R_{5,} R₆, R₁′, R₂′, R₃′, and R₄′ are independently hydrogen, methyl or ethyl,
wherein the cell is a mitotically active cell in vitro in a culture medium comprising 1-1000 nM diaminophenothiazine, and the contacting step comprises culturing the cell in the medium for at least one week.

2. The method of claim 1 wherein R₁, R₂, R₃, R₄, R₅, and R₆ are hydrogen, and R₂, R₅, R₁′, R₂′, R₃′, and R₄′ are independently hydrogen or methyl.

3. The method of claim 1 wherein the diaminophenothiazine is selected from the group consisting of azure A, azure B, azure C, thionin, toluidine blue, methylene blue, new methylene blue, and 1-9-dimethyl methylene blue.

4. The method of claim 1 wherein the diaminophenothiazine is methylene blue.

5. The method of claim 1 wherein the culture medium comprises 10-100 nM methylene blue.

6. The method of claim 1 wherein the contacting step comprises culturing the cell in the medium for at least 4 weeks.

7. The method of claim 1 wherein the medium further comprises an effective amount of a mitochondrial protective agent selected from the group consisting of an N-hydroxylamine, acetyl carnitine, and lipoic acid. Effective is mitochondrial protective amounts.

8. The method of claim 1 wherein the medium further comprises effective amounts of acetyl carnitine and lipoic acid.

9. The method of claim 1 wherein the medium further comprises an effective amount of N-hydroxylamine.

10. A method for delaying cell senescence, the method comprising the step of: contacting a cell specifically determined to be in need of delayed cell senescence with an effective amount of a diaminophenothiazine; wherein the diaminophenothiazine has the structure: 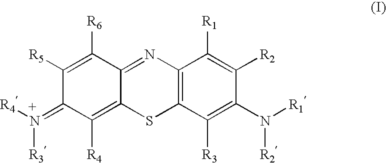
and tautomeric forms thereof, wherein R₁, R₂, R₃, R₄, R₅, R₆, R₁′, R₂′, R₃′, and R₄′ are independently hydrogen, methyl or ethyl,
wherein the cell is in situ in an individual, and the contacting step comprises chronically orally administering to the individual a dosage of 5-500 μg per day of the diaminophenothiazine to provide said effective amount.

11. The method of claim 10 wherein R₁, R₂, R₃, R₄, R₅, and R₆ are hydrogen, and R₂, R₅, R₁′, R₂′, R₃′, and R₄′ are independently hydrogen or methyl.

12. The method of claim 10 wherein the diaminophenothiazine is selected from the group consisting of azure A, azure B, azure C, thionin, toluidine blue, methylene blue, new methylene blue, and 1-9-dimethyl methylene blue.

13. The method of claim 10 wherein the diaminophenothiazine is methylene blue.

14. The method of claim 10 wherein the dosage is administered daily for at least 30 days.

15. The method of claim 10 wherein the individual is over 40 years old.

16. The method of claim 10 wherein the individual is free of diagnosed acute disease or pathology.

17. The method of claim 10 wherein the contacting step additionally comprises chronically orally administering to the individual a mitochondrial protective agent selected from the group consisting of an N-hydroxylamine, acetyl carnitine, and lipoic acid.

18. The method of claim 10 wherein the contacting step additionally comprises chronically orally administering to the individual acetyl carnitine and lipoic acid.

19. The method of claim 10 wherein the contacting step additionally comprises chronically orally administering to the individual an N-hydroxylamine.

20. The method of claim 10 wherein the contacting step additionally comprises chronically orally administering to the individual effective amounts of acetyl carnitine, lipoic acid, and an N-hydroxylamine.

21-36. (canceled)
